(19) United States
(12) Patent Application Publication
(10) Pub. No.: US 2004/0009988 A1
(43) Pub. Date: Jan. 15, 2004

(54) Bioisosteric bensamide derivatives and their use as apob-100 secretion inhibitors

(76) Inventor: Nerina Dodic, Les Ulis (FR)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/296,795

(22) PCT Filed: Jun. 1, 2001
(86) PCT No.: PCT/EP01/06243

(30) Foreign Application Priority Data
Jun. 1, 2000 (GB) 0013383.5

(51) Int. Cl. A61K 31/506; A61K 31/496; A61K 31/4545; C 07D 4 3/04; C 07D 4 1/04

(52) U.S. Cl. 514/252.14; 514/253.01; 514/256; 514/318; 544/295; 544/360; 544/331; 546/194

(57) ABSTRACT

The present invention relates to A compound of formula (I) wherein A, U, V, X, Z, R¹, Y, R² and R³ are defined in the description or a physiologically acceptable salt, solvate or derivative thereof, to compositions and processes for making said compounds and their use in treating conditions ameliorated by an apoB-100 and/or MTP inhibitor.

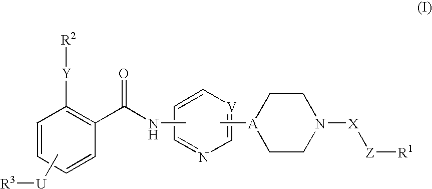

This invention relates to the use of compounds to inhibit hepatic production of apoprotein B-100 (apoB-100) and intestinal production of chylomicrons or apoprotein B-48 (apoB-48) and MTP.

ApoB-100 is the main protein component of low density lipoprotein-cholesterol (LDL-c). High LDL-c plasmatic levels are a major risk factor for atherosclerosis and coronary artery diseases. ApoB-48 is the main protein component of chylomicrons.

The microsomal triglyceride transfer protein (MTP) catalyses the transfer of triglycerides, cholesteryl esters and phosphatidylcholine between small unilamellar vesicles. MTP is expressed in liver and intestine, both organs which produce lipoproteins. MTP is able to lipidate neosynthesized apoB-100 within the liver, and neosynthesized apoB48 within the intestine, therefore leading to the production of triglyceride-rich lipoparticles such as VLDL and chylomicrons respectively. Thus, MTP inhibitors have the potential to decrease LDL-c and triglyceride plasmatic levels, and also intestinal lipid absorption. MTP inhibitors may be used in the treatment of non-insulin dependent diabetes mellitus, coronary heart disease, pancreatitis, hypercholesterolemia, hypertriglyceridemia, hyperlipemia, mixed, dyslipidemia, post-prandial hyperlipemia, atherosclerosis and obesity.

Compounds having apoB-100 and MTP inhibition properties have been described in WO96/40640. PCT/EP99/09320 describes compounds of formula (A) for the treatment of conditions resulting from elevated circulating levels of apoB-100:

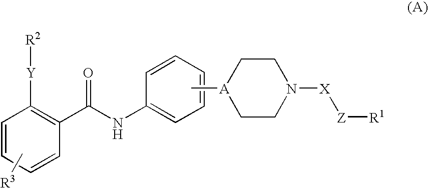

wherein

A represents N or CH;

X is selected from the following groups:

(i) —C₁₋₆alkylene-, optionally containing one or two double bonds and optionally substituted by one or more hydroxy, C₁₋₆ alkyl, C₁₋₆ alkoxy, C₁₋₆acyl or C₁₋₆acyloxy groups,

(ii) oxo, sulfonyl, thioxo,

(iii) —C₁₋₆alkylenecarbonyl-, —C₁₋₆alkylenesulfonyl-, —C₁₋₆alkylenethioxo-,

(iv) —C₂₋₆alkyleneoxy-, —C₂₋₆alkylenethio-, —C₂₋₆alkylene(N—H or N—C₁₋₆alkyl)amino-,

(v) —C₁₋₆alkylenecarboxy-, —C₁₋₆alkylenethioamido-, —C₁₋₆alkylene(N—H or N—C₁₋₆alkyl)carboxamido-, and

(vi) —C₂₋₆alkyleneoxycarbonyl-, —C₂₋₆alkylenethiocarbonyl-, —C₂₋₆ alkylene(N—H or N—C₁₋₆alkyl)aminocarbonyl-;

Z represents a direct link or —C₁₄ alkylene-, optionally containing one double bond and optionally substituted by one or more hydroxy, C₁₋₆ alkyl, C₁₋₆ alkoxy, C₁₋₆ acyl or C₁₋₆ acyloxy groups;

R¹ is selected from the following groups:

(i) hydrogen, C₁₋₃perfluoroalkyl,

(ii) C₆₋₁₀ aryl, C₃₋₈cycloalkyl and fused benz derivatives thereof, C₇₋₁₀polycycloalkyl, C₄₋₈cycloalkenyl, C₇₋₁₀polycycloalkenyl,

(iii) a heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, and

(iv) where either X is C₁₋₆alkylene and Z is a direct link, or Z is C₁₋₆alkylene, R¹ additionally may represent a halogen, cyano, nitro or C₁₋₆acyl group,

wherein, when R¹ contains one or more rings, said rings may each independently bear 0 to 4 substituents independently selected from

(i) halogen, hydroxy, cyano, nitro, formyl, C₁₋₆alkylsulfonylamino,

(ii) C₁₋₆-alkyl, C₃₋₈cycloalkyl, C₁₋₃perfuoroalkyl,

(iii) C₁₋₆alkoxy, methylenedioxy, C₁₋₃perfuoroalkoxy, C₁₋₆alkylthio,

(iv) amino, C₁₋₆alkylamino, di-C₁₋₆alkylamino,

(v) phenyl; phenoxy, phenylthio, halophenylthio, benzyl, benzyloxy,

(vi) hydroxycarbonyl, C₁₋₆alkoxycarbonyl,

(vii) aminocarbonyl, C₁₋₆alkylaminocarbonyl, di-C₁₋₆alkylaminocarbonyl, di-C₁₋₆alkylaminocarbonylC₁₋₆alkoxy, C₁₋₃perfluoroalkylaminocarbonyl,

(viii) C₁₋₆acyl, C₁₋₆acyloxy, C₁₋₆acyloxyC₁₋₆alkyl, C₁₋₆acylamino, and

(ix) an aromatic heterocyclyl consisting of monocyclic radicals, wherein said radicals contain 5-6 ring atoms, wherein said radicals contain a total of from 1-4 ring heteroatoms independently selected from oxygen, nitrogen and sulfur, and where each of the said heterocyclyl groups is optionally substituted by one or more groups independently selected from halogen, C₁₋₄alkyl, C₁₋₄alkoxy, C₁₋₃perfluoroalkyl and C₁₋₃-perfluoroalkoxy;

Y represents a direct or oxy link, —C₁₋₆alkylene-, -oxyC₁₋₆alkylene- or a heterocyclyl consisting of monocyclic radicals, wherein said radicals contain 5 ring atoms, and wherein said radicals contain a total of from 14 ring heteroatoms independently selected from oxygen, nitrogen and sulfur and wherein the ring may be independently saturated, partially unsaturated, or aromatic;

R² represents phenyl, C₃₋₈cycloalkyl, or a heterocyclyl consisting of monocyclic radicals, wherein said radicals contain a total of from 5-6 ring atoms, wherein said radicals contain a total of from 1-4 ring heteroatoms independently selected from oxygen, nitrogen and sulfur, wherein the ring may be independently saturated, partially unsaturated, or aromatic, and where each R² is optionally substituted by one or more groups independently selected from halogen, C₁₋₄alkyl, C₁₋₄alkoxy, C₃₋₈cycloalkyl, C₁₋₃perfluoroalkyl, C₁₋₃perfluoroalkoxy, hydroxycarbonyl, C₁₋₆alkoxycarbonyl, cyano, nitro, C₁₋₄alkylaminosulfonyl;

R³ represents hydrogen or one or more groups independently selected from halogen, C₁₋₄alkyl, C₁₋₄alkoxy, C₁₋₃ perfluoroalkyl or C₁₋₃ perfluoroalkoxy;

or a physiologically acceptable salt, solvate or derivative thereof.

Thus, the present invention provides a compound of formula (I);

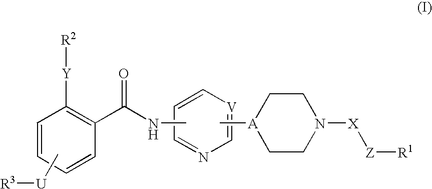

wherein

A represents N or CH;

U represents a direct link, —C₁₋₄alkylene- or —C₀₋₄alkylene-oxy-C₀₋₄alkylene-;

V represents N or CH;

X is selected from the following groups:

(i) —C₁₋₆alkylene-, optionally containing one or two double bonds and optionally substituted by one or more hydroxy, C₁₋₆ alkyl, C₁₋₆ alkoxy, C₁₋₆acyl or C₁₋₆acyloxy groups,

(ii) oxo, sulfonyl, thioxo,

(iii) —C₁₋₆alkylenecarbonyl-, —C₁₋₆alkylenesulfonyl-, —C₁₋₆alkylenethioxo-,

(iv) —C₂₋₆alkyleneoxy-, —C₂₋₆alkylenethio-, —C₂₋₆alkylene(N—H or N—C₁₋₆alkyl)amino-,

(v) —C₁₋₆-alkylenecarboxy-, —C₁₋₆alkylenethioamido-, —C₁₋₆alkylene(N—H or N—C₁₋₆alkyl)carboxamido-, and

(vi) —C₂₋₆alkyleneoxycarbonyl-, —C₂₋₆alkylenethiocarbonyl-, —C₂₋₆ alkylene(N—H or N—C₁₋₆alkyl)aminocarbonyl-;

Z represents a direct link or —C₁₋₆ alkylene-, optionally containing one double bond and optionally substituted by one or more hydroxy, C₁₋₆ alkyl, C₁₋₆ alkoxy, C₁₋₆ acyl or C₁₋₆ acyloxy groups;

R¹ is selected from the following groups:

(i) hydrogen, C₁₋₃perfluoroalkyl,

(ii) C₆₋₁₀ aryl, C₃₋₈cycloalkyl and fused benz derivatives thereof, C₇₋₁₀polycycloalkyl, C₄₋₈cycloalkenyl, C₇₋₁₀polycycloalkenyl,

(iii) a heterocyclyl selected from the group consisting of monocyclic radicals and fused polycyclic radicals, wherein said radicals contain a total of from 5-14 ring atoms, wherein said radicals contain a total of from 14 ring heteroatoms independently selected from oxygen, nitrogen and sulfur, and wherein individual rings of said radicals may be independently saturated, partially unsaturated, or aromatic, and

(iv) where either X is C₁₋₆alkylene and Z is a direct link, or Z is C₁₋₆alkylene, R¹ additionally may represent a halogen, cyano, nitro or C₁₋₆acyl group;

wherein, when R¹ contains one or more rings, said rings may each independently bear 0 to 4 substituents independently selected from:

(i) halogen, hydroxy, cyano, nitro, formyl, C₁₋₆alkylsulfonylamino,

(ii) C₁₋₆alkyl, C₃₋₆cycloalkyl, C₁₋₃perfluoroalkyl,

(iii) C₁₋₆alkoxy, methylenedioxy, C₁₋₃perfluoroalkoxy, C₁₋₆alkylthio,

(iv) amino, C₁₋₆alkylamino, di-C₁₋₆alkylamino,

(v) phenyl, phenoxy, phenylthio, halophenylthio, benzyl, benzyloxy,

(vi) hydroxycarbonyl,C₁₋₆alkoxycarbonyl,

(vii) aminocarbonyl, C₁₋₆alkylaminocarbonyl, di-C₁₋₆alkylaminocarbonyl, di-C₁₋₆alkylaminocarbonylC₁₋₆alkoxy, C₁₋₃perfluoroalkylaminocarbonyl,

(viii) C₁₋₆acyl, C₁₋₆acyloxy, C₁₋₆acyloxyC₁₋₆alkyl, C₁₋₆acylamino, and

(ix) an aromatic heterocyclyl consisting of monocyclic radicals, wherein said radicals contain 5-6 ring atoms, wherein said radicals contain a total of from 14 ring heteroatoms independently selected from oxygen, nitrogen and sulfur, and where each of the said heterocyclyl groups is optionally substituted by one or more groups independently selected from halogen, C₁₋₄alkyl, C₁₋₄alkoxy, C₁₋₃perfuoroalkyl and C₁₋₃perfuoroalkoxy;

Y represents a direct or oxy link, —C₁₋₆alkylene-, -oxyC₁₋₆alkylene- or a heterocyclyl consisting of monocyclic radicals, wherein said radicals contain 5 ring atoms, and wherein said radicals contain a total of from 1-4 ring heteroatoms independently selected from oxygen, nitrogen and sulfur and wherein the ring may be independently saturated, partially unsaturated, or aromatic;

R² represents phenyl, C₃₋₈cycloalkyl, or a heterocyclyl consisting of monocyclic radicals, wherein said radicals contain a total of from 5-6 ring atoms, wherein said radicals contain a total of from 1-4 ring heteroatoms independently selected from oxygen, nitrogen and sulfur, wherein the ring may be independently saturated, partially unsaturated, or aromatic, and where each R² is optionally substituted by one or more groups independently selected from halogen, C₁₋₄alkyl, C₁₋₄alkoxy, C₃₋₈cycloalkyl, C₁₋₃perfuoroalkyl, C₁₋₃perfuoroalkoxy, hydroxycarbonyl, C₁₋₆alkoxycarbonyl, cyano, nitro and C₁₋₄alkylaminosulfonyl;

R³ is selected from the following groups:

i) hydrogen or C₁₋₃perfluoroalkyl,

ii) phenyl or a heterocyclyl consisting of monocyclic radicals, wherein said radicals contain a total of 5-6 ring atoms, wherein said radicals contain a total of from 1-4 ring heteroatoms selecetd from oxygen, nitrogen or sulfur, and wherein the ring may be saturated, partially unsaturated or aromatic,

iii) cyano, hydroxycarbonyl, C₁₋₆alkoxycarbonyl, aminocarbonyl, C₁₋₆alkylaminocarbonyl or C₁₋₆dialkylaminocarbonyl, with the proviso that U may not represent —C₁₋₄alkylene-oxy-,

iv) halogen, amino, C₁₋₆alkylamino or C₁₋₆dialkylamino, with the proviso that U may not represent —C₀₋₄alkylene-oxy-C₀₋₁alkylene,

wherein, when R³ contains one or more rings, said rings may each independently bear 0 to 4 substituents independently selected from C₁₋₆ alkyl, C₁₋₆alkoxy, hydroxy and halogen;

or a physiologically acceptable salt, solvate or derivative thereof.

Suitable physiologically acceptable salts of the compounds of general formula (I) include acid addition salts formed with pharmaceutically acceptable organic and inorganic acids for example, citrates, hydrochlorides, hydrobromides, or sulphates. Particularly preferred salts are citrates or hydrochloride salts.

The solvates may, for example, be hydrates.

References hereinafter to a compound according to the invention include both compounds of formula (I) and their physiologically acceptable salts together with physiologically acceptable solvates.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), an alkyl or alkylene group containing one double bond constitutes an alkenyl or alkenylene group respectively. Such groups include both straight and branched chain hydrocarbon groups, e.g. prop-2-enyl and but-2-enyl.

Referring to general formula (I), a halogen atom may be a fluorine, chlorine, bromine or iodine atom.

Referring to the general formula (I), reference to heterocyclyl, unless otherwise defined, means any single ring or fused ring system containing at least one ring heteroatom independently selected from O, N and S. Thus, a polycyclic fused ring system containing one or more carbocyclic fused saturated, partially unsaturated, or aromatic rings (usually benz rings) is within the definition of heterocyclyl so long as the system also contains at least one fused ring which contains at least one of the aforementioned heteroatoms. As a substituent, such heterocyclyls may be attached to the remainder of the molecules from either a carbocyclic (e.g. benz) ring or from a heterocyclic ring.

Referring to the general formula (I), reference to R¹ and R³ as containing one or more rings is intended to mean any single or fused cyclic moiety or moieties attached to Z or U respectively. The rings may be carbocyclic or heterocyclic, saturated or partially unsaturated, and aromatic or non-aromatic.

Reference to a polycyclic ring system or radical means that all rings in the system are fused.

Referring to the general formula (I), aryl means that the ring or substituent is carbocyclic and includes phenyl and naphthyl.

Referring to the general formula (I), acyl refers to aliphatic or cyclic hydrocarbons attached to a carbonyl group through which the substituent bonds.

Referring to the general formula (I), methylenedioxy refers to a x,x+1-methylenedioxy group, where x and x+1 are integers which represent the substitiution pattern on the ring, e.g. 3,4-methylenedioxy.

Referring to the general formula (I), C₁₋₃perfuoroalkyl or C₁₋₃perfuoroalkoxy includes compounds such as trifluoromethyl and trifluoromethoxy.

Preferably, A represents N.

X is suitably —C₁₋₆alkylene-, optionally containing by one double bond, e.g. methylene, ethylene, propylene, prop-2-enylene or but-2-enylene, oxo, sulfonyl, —C₂₋₆alkyleneoxy-, e.g. ethyleneoxy or propyleneoxy, —C₁₋₆alkylenecarboxy-, e.g. methylenecarboxy or —C₁₋₆alkylene(N—H or N—C₁₋₆alkyl)carboxamido-, e.g. methylene(N—H)carboxamido.

X is equally suitably —C₁₋₆alkylene- e.g. methylene, propylene or prop-2-enylene, or —C₁₋₆alkylene(N—H or N—C₁₋₆alkyl)carboxamido-, e.g. methylene(N—H)carboxamido. As a preferred aspect, X is a methylene, propylene, prop-2-enylene or methylene(N—H)carboxamido. More preferably, X is methylene.

Z is suitably a direct link or —C₁₋₆alkylene-, e.g. methylene or ethylene. Z is most suitably a direct link.

R¹ is suitably selected from the following groups

(i) hydrogen, cyano, C₁₋₃perfuoroalkyl, e.g. trifluoromethyl,

(ii) optionally substituted phenyl, where optional substitution is effected by one or two groups independently selected from C₁₋₆alkyl, e.g. methyl, cyano, halogen, e.g. fluoro, C₁₋₆alkoxy, e.g. methoxy, C₁₋₃perfuoroalkyl, e.g. trifluoromethyl, hydroxycarbonyl, C₁₋₄alkoxycarbonyl, e.g. methoxycarbonyl, aminocarbonyl, methylenedioxy, nitro, C₁₋₆ acyl, e.g acetyl, phenyl, or an optionally substituted aromatic heterocycyl consisiting of monocyclic radicals and fused polycyclic radicals, wherein said radicals contain a total of 5 ring atoms, e.g. oxadiazolyl, where optional substitution is effected by C₁₋₄ alkyl, e.g. methyl, or C₁₋₃perfluoroalkyl, e.g. trifluoromethyl, or

(iii) an optionally substituted aromatic heterocyclyl consisiting of monocyclic radicals and fused polycyclic radicals, wherein said radicals contain a total of from 5-10 ring atoms, e.g. indolyl, pyrrolyl, thienyl, furanyl, imidazolyl, pyrazolyl, thiazolyl, pyridyl or pyrazinyl, where optional substitution is effected by C₁₋₄ alkyl, e.g. methyl, or halogen, e.g. fluorine, or cyano.

Where R¹ is a substituted phenyl group, substitution is suitably in the 3-position.

When R¹ is an optionally substituted aromatic heterocyclyl, R¹ is preferably an optionally substituted pyrrolyl, more preferably, a 2-pyrrolyl group, where optional substitution is suitably effected by a methyl group.

R¹ is preferably selected from hydrogen, substituted phenyl, where substitution is effected by cyano or a methyl substituted [1,2,4]-oxadiazol-5-yl group, or a pyrrolyl or furanyl group.

R¹ is most preferably pyrrolyl, or phenyl substituted by 3-methyl-[1,2,4]-oxadiazol-5-yl.

X-Z is suitably methylene and R¹ is suitably phenyl or a 5-membered aromatic heterocyclyl, e.g. pyrrolyl or furanyl, where each R¹ is optionally substitued by one or more groups independently selected from C₁₋₆ alkyl, e.g. methyl, cyano, halogen, e.g. fluoro, C₁₋₆alkoxy, e.g. methoxy, or trifluoromethyl.

X-Z is equally suitably —C₁₋₆alkylene-, e.g. methylene or propylene, C₂₋₆alkenylene, e.g. prop-2-enylene, or methylene(N—H)carboxyamido and R¹ is suitably hydrogen.

As a most preferred substitution pattern, —X-Z-R¹ is suitably methyl, n-propyl, prop-2-enyl, aminocarbonylmethyl, pyrrolylmethyl or phenylmethyl substituted by 3-cyano or 3-(3-methyl-[1,2,4]-oxadiazol-5-yl).

Y is suitably a direct link, a 2,5-substituted oxazolyl group, or —(CH₂)ₙ—O—, where n is an integer from 0-3. More suitably, Y is a direct or oxy link. Preferably Y is a direct link.

R² is suitably cyclohexyl, a 5-6 membered aromatic heterocyclyl, e.g. pyrrolyl or pyridyl, or a phenyl group optionally substituted by one or two groups independently selected from halogen, e.g. fluoro or chloro, C₁₋₄alkyl, e.g. methyl, ethyl or isopropyl, C₁₋₄ alkoxy, e.g. methoxy, or trifluoromethyl groups, where substitution is suitably in one or two of the 2-, 3-, or 4-positions on the phenyl ring. Preferably, R² is a phenyl group substituted by a trifluoromethyl group, most preferably in the 4-position. Equally preferably, R² is a phenyl group substituted by an isopropyl group, most preferably in the 4-position.

Preferably, Y—R² is a phenyl group substituted by a trifluoromethyl or isopropyl group, most preferably in the 4-position.

Referring to the heteroaromatic ring containing V and N radicals in compounds of formula (I), the pendant radical defined by A and the aminocarbonyl group are suitably disposed para to each other and, more suitably, are disposed in the 2- and 5-position respectively to the ring N radical.

V is preferably CH.

U is suitably a direct link, C₁₋₄alkylene e.g. methylene, ethylene or isopropylene, oxy, methyleneoxy or oxymethylene.

Preferably, U is a direct link, methylene, isopropylene or oxymethylene.

R³ is suitably hydrogen, C₁₋₃ perfluoroalkyl, e.g. trifluoromethyl, C₁₋₆dialkylamino e.g. dimethylamino, phenyl, an aromatic heterocylyl, e.g, pyridyl, pyrrollyl, imidazolyl, thiazolyl and oxadiazolyl, or a saturated or partially unsaturated heterocylyl, e.g. piperidyl.

R³ is preferably hydrogen or trifluoromethyl.

U—R³ is suitably hydrogen, halogen, e.g. fluoro or chloro, C₁₋₄alkyl, e.g. methyl or isopropyl, C₁₋₄alkoxy, e.g. methoxy or C₁₋₃perfluoroalkyl, e.g. trifluoromethyl, C₁₋₆dialkylamino, e.g. methylenedialkylamino.

U—R³ is preferably hydrogen, methyl, isopropyl, methoxy or trifluoromethyl.

U—R³ is suitably 5- or 6- substituted, relative to group Y, preferably 6- substituted.

Particularly preferred compounds of the invention include those in which each variable in formula (I) is selected from the preferred groups for each variable. Even more preferable compounds of the invention include those where each variable in formula (I) is selected from the more preferred or most preferred groups for each variable.

A suitable sub-group of a compound of formula (I) is represented by formula (Ia)

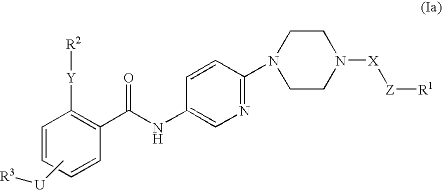

wherein

U—R³ is suitably hydrogen, halogen, C₁₋₄ alkyl, C₁₋₄alkoxy or C₁₋₃perfluoroalkyl;

X is suitably —C₁₋₆alkylene-, optionally containing one double bond, oxo, sulfonyl, —C₂₋alkyleneoxy-, —C₁₋₆alkylenecarboxy- or —C₁₋₆alkylene(N—H or N—C₁₋₆alkyl)carboxamido-;

Z represents a direct link or —C₁₋₆alkylene-;

R¹ represents one of the following groups:

(i) hydrogen,

(ii) optionally substituted phenyl, where optional substitution is effected by one or two groups independently selected from C₁₋₆ alkyl, cyano, halogen, C₁₋₆alkoxy; C₁₋₃perfuoroalkyl, hydroxycarbonyl, C₁₋₄alkoxycarbonyl, aminocarbonyl, C₁₋₃perfluoroalkylaminocarbonyl, methylenedioxy, nitro, C₁₋₆acyl, phenyl, or an optionally substituted aromatic heterocyclyl consisiting of monocyclic radicals and fused polycyclic radicals, wherein said radicals contain a total of 5 ring atoms, where optional substitution is effected by C₁₋₄ alkyl, or C₁₋₃perfluoroalkyl,

(iii) an optionally substituted aromatic heterocycyl consisiting of monocyclic radicals and fused polycyclic radicals, wherein said radicals contain a total of from 5-10 ring atoms, where optional substitution is effected by C₁₋₄ alkyl, or C₁₋₃perfluoroalkyl; or

(iv) C₁₋₆aminocarbonyl, or

(iv) where either X is C₁₋₆alkylene and Z is a direct link, or Z is C₁₋₆alkylene, R¹ additionally may represent a cyano group;

Y represents a direct or oxy link, a 5-membered aromatic heterocyclyl group, —C₁₋₆alkylene- or -oxyC₁₋₆alkylene-;

R² represents phenyl substituted by one or two groups independently selected from halogen, trifluoromethyl, C₁₋₄alkyl and C₁₋₄alkoxy groups;

or a physiologically acceptable salt, solvate or derivative thereof.

A further suitable sub-group of a compound of formula (I) is represented by formula (Ib)

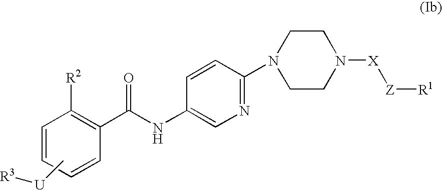

wherein

U—R³ is suitably hydrogen, halogen, C₁₋₄ alkyl, C₁₋₄alkoxy or C₁₋₃perfluoroalkyl;

X-Z-R¹ represents C₁₋₆alkyl, C₂₋₆alkenyl, aminocarbonylmethyl, an aromatic 5-membered heterocyclylmethyl containing 1-4 heteroatoms chosen from oxygen, nitrogen and sulfur or phenylmethyl substituted by cyano or a methyl-substituted oxadiazolyl;

R² represents phenyl substituted by one or two groups independently selected from halogen, trifluoromethyl, C₁₋₄alkyl and C₁₋₄alkoxy groups;

or a physiologically acceptable salt, solvate or derivative thereof.

A yet further suitable sub-group of the invention is represented by a compound of formula (Ic)

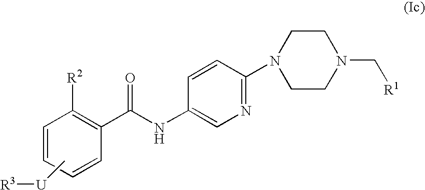

wherein

U—R³ is suitably hydrogen, halogen, C₁₋₄ alkyl, C₁₋₄alkoxy or C₁₋₃perfluoroalkyl;

R¹ represents phenyl optionally substitued by one or two groups independently selected from C₁₋₆ alkyl, cyano, halogen, C₁₋₆ alkoxy, trifluoromethyl, hydroxycarbonyl and C₁₋₆alkoxycarbonyl;

R² represents phenyl substituted in the 4-position by a halogen, trifluoromethyl, C₁₋₄alkyl or C₁₋₄alkoxy group;

or a physiologically acceptable salt, solvate or derivative thereof.

It will be clear that references herein to a compound of formula (I) apply equally to a compound of formula (Ia)-(Ic).

Suitable compounds according to the invention include:

4′-isopropyl-6-methyl-biphenyl-2-carboxylic acid [2-(4-carbamoylmethyl-piperazin-1-yl)-pyridin-5-yl]-amide;

6-methyl-4′-trifluoromethyl-biphenyl-2-carboxylic acid [2-(4-carbamoylmethyl-piperazin-1-yl)-pyridin-5-yl]-amide;

4′-isopropyl-6-methoxy-biphenyl-2-carboxylic acid [2-(4-carbamoylmethyl-piperazin-1-yl)-pyridin-5-yl]-amide;

4′,6-diisopropyl-biphenyl-2-carboxylic acid [2-(4-carbamoylmethyl-piperazin-1-yl)-pyridin-5-yl]-amide;

4′-isopropyl-6-methyl-biphenyl-2-carboxylic acid [2-(4-(1H-pyrrol-2-ylmethyl)piperazin-1-yl)-pyridin-5-yl]-amide;

6-methyl-4′-trifluoromethyl-biphenyl-2-carboxylic acid [2-(4-(1H-pyrrol-2-ylmethyl)piperazin-1-yl)-pyridin-5-yl]-amide;

4′-isopropyl-6-methoxy-biphenyl-2-carboxylic acid [2-(4-(1H-pyrrol-2-ylmethyl)piperazin-1-yl)-pyridin-5-yl]-amide;

4′-6-diisopropyl-biphenyl-2-carboxylic acid [2-(4-(1H-pyrrol-2-ylmethyl)piperazin-1-yl)-pyridin-5-yl]-amide;

4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;

4′-isopropyl-6-methyl-biphenyl-2-carboxylic acid [2-(4-(3-(3-methyl-[1,2,4]oxadiazol-5-yl)-benzyl)-piperazin-1-yl)-pyridin-5-yl]-amide;

6-methyl-4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;

4′-isopropyl-6-methoxy-biphenyl-2-carboxylic acid [2-(4-(3-(3-methyl-[1,2,4]oxadiazol-5-yl)-benzyl)-piperazin-1-yl)-pyridin-5-yl]-amide;

4′,6-diisopropyl-biphenyl-2-carboxylic acid [2-(4-(3-(3-methyl-[1,2,4]oxadiazol-5-yl)-benzyl)-piperazin-1-yl)-pyridin-5-yl]-amide;

4′-trifluoromethyl-biphenyl-2-carboxylic acid [2-(4-(furan-2-ylmethyl)piperazin-1-yl)-pyridin-5-yl]-amide;

4′,6-diisopropyl-biphenyl-2-carboxylic acid [2-(4-(3-cyano-benzyl)-piperazin-1-yl)-pyridin-5-yl]amide;

4′-trifluoromethyl-biphenyl-2-carboxylic acid [2-(4-(3-cyano-benzyl)-piperazin-1-yl)-pyrimydin-5-yl]-amide;

4′-trifluoromethyl-biphenyl-2-carboxylic acid [2-(4-(3-cyano-benzyl)-piperazin-1-yl)-pyridin-5-yl]-amide;

4′-isopropyl-6-methoxy-biphenyl-2-carboxylic acid [2-(4-(3-cyano-benzyl)- piperazin-1-yl)-pyridin-5-yl )-amide;

4′-isopropyl-6-methyl-biphenyl-2-carboxylic acid [2-(4-(3-cyano-benzyl)-piperazin-1-yl)-pyridin-5-yl]-amide;

6-methyl-4′-trifluoromethyl-biphenyl-2-carboxylic acid [2-(4-(3-cyano-benzyl)-piperazin-1-yl)-pyridin-5-yl]-amide;

4′-isopropyl-5-methyl-biphenyl-2-carboxylic acid [2-(4-(3-cyano-benzyl)-piperazin-1-yl)-pyridin-5-yl]-amide;

5-methyl-4′-trifluoromethyl-biphenyl-2-carboxylic acid [2-(4-(3-cyano-benzyl)-piperazin-1-yl)-pyridin-5-yl]-amide;

4′,6-diisopropyl-biphenyl-2-carboxylic acid-[2-(4-propyl-piperazin-1-yl)-pyridin-5-yl]-amide;

4′,6-diisopropyl-biphenyl-2-carboxylic acid-[2-(4-methyl-piperazin-1-yl)-pyridin-5-yl]-amide;

4′,6-diisopropyl-biphenyl-2-carboxylic acid-[2-(4-(propen-2-y)l-piperazin-1-yl)-pyridin-5-yl]-amide;

4′,6-Diisopropyl-biphenyl-2-carboxylic acid-[2-(4-(isopropyl)-piperazin-1-yl)-pyridin-5-yl]-amide;

or a physiologically acceptable salt, solvate or derivative thereof.

Preferred compounds of the invention include:

4′-isopropyl-6-methyl-biphenyl-2-carboxylic acid [2-(4-carbamoylmethyl-piperazin-1-yl)-pyridin-5-yl]-amide;

6-methyl-4′-trifluoromethyl-biphenyl-2-carboxylic acid [2-(4-carbamoylmethyl-piperazin-1-yl)-pyridin-5-yl]-amide;

4′-isopropyl-6-methoxy-biphenyl-2-carboxylic acid [2-(4-carbamoylmethyl-piperazin-1-yl)-pyridin-5-yl]-amide;

4′-isopropyl-6-methyl-biphenyl-2-carboxylic acid [2-(4-(1H-pyrrol-2-ylmethyl)piperazin-1-yl)-pyridin-5-yl]-amide;

6-methyl-4′-trifluoromethyl-biphenyl-2-carboxylic acid [2-(4-(1H-pyrrol-2-ylmethyl)piperazin-1-yl)-pyridin-5-yl]-amide;

4′-isopropyl-6-methoxy-biphenyl-2-carboxylic acid [2-(4-(1H-pyrrol-2-ylmethyl)piperazin-1-yl)-pyridin-5-yl]-amide;

4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;

4′-isopropyl-6-methyl-biphenyl-2-carboxylic acid [2-(4-(3-(3-methyl-[1,2,4]oxadiazol-5-yl)-benzyl)-piperazin-1-yl)-pyridin-5-yl]-amide;

6-methyl-4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;

4′-isopropyl-6-methoxy-biphenyl-2-carboxylic acid [2-(4-(3-(3-methyl-[1,2,4]oxadiazol-5-yl)-benzyl)-piperazin-1-yl)-pyridin-5-yl]-amide;

4′,6-diisopropyl-biphenyl-2-carboxylic acid-[2-(4-propyl-piperazin-1-yl)-pyridin-5-yl]-amide;

4′,6-diisopropyl-biphenyl-2-carboxylic acid-[2-(4-methyl-piperazin-1-yl)-pyridin-5-yl]-amide;

4′,6-diisopropyl-biphenyl-2-carboxylic acid-[2-(4-(propen-2-y)l-piperazin-1-yl)-pyridin-5-yl]-amide;

or a physiologically acceptable salt, solvate or derivative thereof.

The term “physiologically functional derivative” as used herein refers to any physiologically acceptable derivative of a compound of the present invention, for example, an ester or amide, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

The compounds of the invention are inhibitors of hepatic production of apoB-100 and MTP and are thus of use in the treatment of conditions ameliorated by an apoB-100 and/or MTP inhibitor.

The ability of the compounds of this invention to inhibit human MTP activity is measured by an in vitro assay where MTP tranfers 3H-triolein between phosphatidylcholine liposomes. The specificity of the compounds of the invention is established by comparing the effects on apoB-100 and apoprotein A-1 production. A specificity of at least 100 is preferred.

The in vivo profile of the compounds is determined by acute oral administration of the compounds of the invention to DBA/2 mice and Wistar rats. Potency of the active compounds is evaluated by measuring plasmatic lipids (total cholesterol, triglyceride, LDL cholesterol and HDL cholesterol) and apoproteins (apoB-100, apoB-48 and apoA-1).

The compounds of the invention are potent and specific inhibitors of hepatic production of apoB-100 and MTP, which furthermore exhibit good oral bioavailability and duration of action.

Compounds of the invention are of use in the treatment of atherosclerosis, pancreatitis, non-insulin dependent diabetes mellitus (NIDDM), coronary heart diseases and obesity.

Compounds of the invention are also useful in lowering serum lipid levels, cholesterol and/or triglycerides, and are of use in the treatment of hyperlipidemia, post-prandial hyperlipemia, mixed dyslipidemia, hyperlipoproteinemia, hypercholesterolemia and/or hypertriglyceridemia.

The invention therefore provides a compound of formula (I) or a physiologically acceptable salt, solvate or derivative thereof for use in therapy, in particular in human medicine.

There is also provided as a further aspect of the invention the use of a compound of -formula (I) or a physiologically acceptable salt, solvate or derivative thereof in the preparation of a medicament for use in the treatment of conditions ameliorated by an apoB-100 and/or MTP inhibitor.

In an alternative or further aspect, there is provided a method for the treatment of a mammal, including man, comprising administration of an effective amount of a compound of formula (I) or a physiologically acceptable salt, solvate or derivative thereof in particular in the treatment of conditions ameliorated by an apoB-100 and/or MTP inhibitor.

It will be appreciated that reference to treatment is intended to include prophylaxis as well as the alleviation of established symptoms. Compounds of formula (I) may be administered as the raw chemical but the active ingredient is preferably presented as a pharmaceutical formulation.

Accordingly, the invention also provides a pharmaceutical composition which comprises at least one compound of formula (I) or a physiologically acceptable salt, solvate or derivative thereof and formulated for administration by any convenient route. Such compositions are preferably in a form adapted for use in medicine, in particular human medicine, and can conveniently be formulated in a conventional manner using one or more pharmaceutically acceptable carriers or excipients.

Thus compounds of formula (I) may be formulated for oral, buccal, parenteral, transdermal, topical (including ophthalmic and nasal), depot or rectal administration or in a form suitable for administration by inhalation or insufflation (either through the mouth or nose).

The compounds of formula (I) may, if desired, be administered with one or more therapeutic agents and formulated for administration by any convenient route in a conventional manner. Appropriate doses will be readily appreciated by those skilled in the art. For example, the compounds of formula (I) may be administered in combination with an HMG CoA reductase inhibitor.

A compound of formula (I), or a physiologically, acceptable salt, solvate or derivative thereof, may be prepared by the general methods outlined hereafter. In the following description, the groups A, U, V, X, Y, Z, R¹, R² and R³ are as previously defined for compounds of formula (I), unless specified otherwise.

According to a general process (A), a compound of formula (I) may be prepared by reacting a compound of formula (II) with a compound of formula R¹-Z-X-L

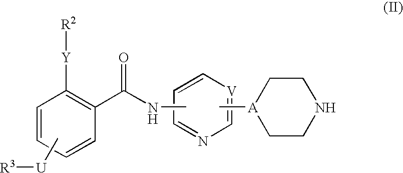

where L represents a suitable halide leaving group, e.g. chloride or bromide, under standard displacement conditions, or where X is an oxo group, L may additonally represent a hydroxy group, the reaction being effected under standard acid and amine coupling conditions.

A compound of formula (II) may be prepared by reaction of a compound of formula (III) with a compound of formula (IV)

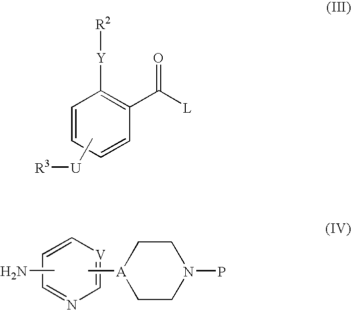

where L is defined above and P is a suitable amine protecting group, e.g. tert-butoxycarbonyl (Boc) or benzyl, under standard coupling conditions for an acid and amine coupling, followed by deprotection of the protecting group under suitable conditions, e.g. acidic removal of a Boc group or hydrogenation of the benzyl group.

A compound of formula (IV), where A represents N, may be prepared by the two step reaction of a compound of formula (V)

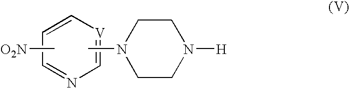

comprising incorporation of the protecting group P using standard methodology followed by reduction of the nitro group, e.g. under hydrogenation conditions or by SnCl2 reduction.

A compound of formula (IV) may alternatively be prepared by reaction of a compound (Va) with a compound (Vb)

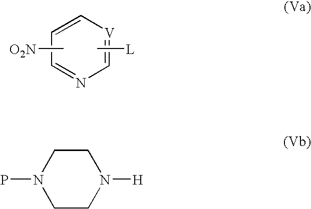

where L is a suitable leaving group such as chloride or bromide and P is a suitable N-protecting group as descibed above, followed by reduction of the nitro group, e.g. under hydrogenation conditions or by SnCl2 reduction.

A compound of formula (IV), where A represents CH, may be prepared from a compound of formula (VI)

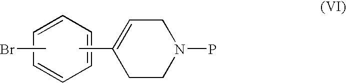

where P is defined above, by reaction with a suitable a compound of formula H₂N—P′ where P′ is a suitable protecting group which is labile under hydrogenation conditions, such as a benzyl group, using a suitable coupling agent or agents such as tris(dibenzylidene acetone)dipalladium, 2,2′-bis(diphenylphosphino)-1,1′-binaphthyl (binap) and sodium tert-butoxide in a suitable solvent such as toluene, followed by removal of the protecting group and reduction of the double bond under hydrogenation conditions.

According to a second method (B), compounds of formula (I) may be prepared by reaction of compounds of formula (III) and compounds of formula (VII)

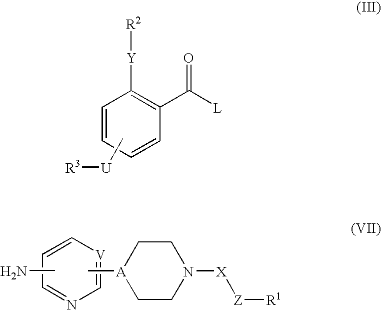

where L is defined above, under standard coupling conditions.

Compounds of formula (VII) may be prepared by reaction of a compound of formula (V) with a compound of formula R¹-Z-X-L, where L is defined above, followed by reduction of the nitro group under hydrogenation or reductive tin chloride conditions.

Alternatively, compounds of formula (VII) may be prepared from a compound of formula (VIIa)

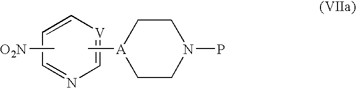

comprising deprotection of N-protecting group P under standard conditions, followed by reaction of the resulting compound with R¹-Z-X-L, as defined above, followed by reduction of the nitro group under standard conditions.

According to a third process (C), a compound of formula (I) where Y is —O—C₁₋₄alkylene- may be prepared by reaction of a compound of formula (VIII) with a compound of formula R²—C₁₋₄alkylene-L, where L is defined above,

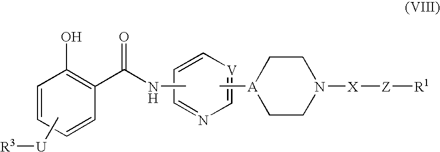

Compounds of formula (VIII) may be prepared according to the process outlined in process B.

According to a fourth general process (D), a compound of formula (I), where at least part of X represents an alkylene link to the piperidine or piperazine group, may be prepared by reacting a compound of formula (II) with a compound of formula (IX)

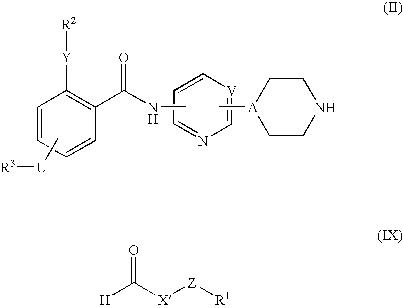

where X′ represents X minus a methylene group, under standard reductive amination conditions, e.g. using sodium triacetoxyborohydride in a solvent such as dichloroethane.

According to a fifth process (E), a compound of formula (I) may be prepared from a different compound of formula (I), using standard techniques well known in the art. For example, compounds of formula (I) where R¹ comprises a group containing an amide group may be prepared from the compound of formula (I) where the corresponding position comprises a carboxylic acid group, which in turn may be prepared from the compound of formula (I) where the corresponding position comprises a carboxylic ester group. Well known methods in the art may be employed to facilitate the transformation of an ester to an acid and then to an amide.

A compound of formula (III), where Y is a direct link, R² is a phenyl or an aromatic heterocyclyl and L is a hydroxy group, may be prepared firstly by coupling a boronic acid with a suitable leaving group, represented by a compound of formula (X) and a compound of formula (XI)

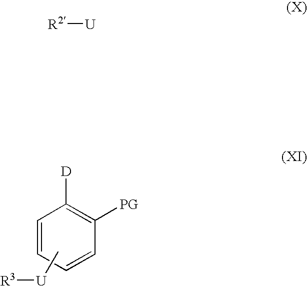

where R²′ represents phenyl or an aromatic heterocyclyl, PG represents a protected carboxylic acid and A and D represent either the boronic acid or the suitable leaving group, such as triflate or bromide, followed by deprotection of the protecting group under standard conditions, such as base removal of an ester group. Where L represents a halide leaving group, the carboxylic acid product can be treated with a suitable reagent, such as thionyl chloride, to give the corresponding chloride leaving group.

Where R¹ is a phenyl, substituted by an aromatic heterocyclyl, the aromatic heterocyclyl may be introduced by any well known methods in the art. For instance, where the substituent is a methyl substituted oxadiazole, this may be formed by treatment of a suitable benzamide derivative with a suitable reagent, such as dimethylacetamide dimethylacetal at elevated temperature, followed by cyclisation of the intermediate compound with hydoxylamine.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

Compounds of formula R¹-Z-X-L, (III), (V), (Va), (Vb) (VI), (IX), (X), (Xa) and (XI) are known or may be prepared by standard methods well known in the art and/or herein described.

Physiologically acceptable salts may also be prepared from other salts, including other physiologically acceptable salts, of the compound of formula (I) using conventional methods.

The compounds of formula (I) may readily be isolated in association with solvent molecules by crystallisation from or evaporation of an appropriate solvent to give the corresponding solvates.

When a specific enantiomer of a compound of general formula (I) is required, this may be obtained for example by resolution of a corresponding enantiomeric mixture of a compound of formula (I) using conventional methods.

Thus, in one example an appropriate optically active acid may be used to form salts with the enantiomeric mixture of a compound of general formula (I). The resulting mixture of isomeric salts may be separated, for example, by fractional crystallisation into the diastereoisomeric salts from which the required enantiomer of a compound of general formula (I) may be isolated by conversion into the required free base.

Alternatively, enantiomers of a compound of general formula (I) may be synthesised from the appropriate optically active intermediates using any of the general processes described herein.

The invention is further illustrated by the following intermediates and examples. All temperatures are in degrees centigrade.

Abbreviations:

MS—LCMS mass spectrography, HOBt-1-Hydroxybenzotriazole, AcOEt—Ethyl acetate, EDCl-1-(3-dimethylaminopropyl)-3-ethylcarbodiimide hydrochloride, BINAP-2,2′-bis(diphenylphosphino)-1,1′-binaphthyl, THF—Tetrahydrofuran, MeOH—Methanol, EtOH—Ethanol, Et₃N—Triethylamine

Intermediate 1

4′-6-Diisopropyl-biphenyl-2-carboxylic acid methyl ester

To a stirred solution of 3-isopropyl-2-(trifluoro-methanesulfonyloxy)-benzoic acid methyl ester (2.3 g) in toluene (15 mL) was added LiCl (0.88 g) and Pd(PPh₃)₄ (0.402 g). After 10 minutes at room temperature, a 2M solution of Na₂CO₃ (7 mL) was added followed by 4-isopropylphenyl boronic acid (1.43 g) in EtOH (10 mL). The resulting mixture was heated under reflux during 6 hours and then cooled to room temperature. After decantation, the organic phase was diluted, washed with water, dried over Na₂SO₄, filtered and concentrated under reduced pressure. The title compound was obtained as a brown oil (2.07 g).

GC/MS: m/z 296 (M+).

Intermediate 2

4′-6-Diisopropyl-biphenyl-2-carboxylic acid

To a stirred solution of 4′-6-diisopropyl-biphenyl-2-carboxylic acid methyl ester (2.07 g) in ethanol (10 mL) was added NaOH (solution 1N, 21 mL) and the mixture was heated under reflux overnight. After concentration under reduced pressure, the residue was taken in water and the aquous phase was washed with diethyle oxyde and then made acidic with HCl (solution 1N). The aquous phase was extracted with diethyle oxyde and the organic phase was dried over Na₂SO₄, filtered and concentrated under reduced pressure. After crystallisation from MeOH/H₂O, the title compound was obtained as white crystals (1.6 g).

m.p.: 123-125° C.

Intermediate 3

5-Nitro-2-piperazinyl-pyridine

To a solution of piperazine (21.18 g) and potassium carbonate (6.9 g) in DMF (250 mL), was added dropwise a solution of 2-bromo-5-nitro-pyridine (10 9) in DMF (50 mL). The mixture was stirred at room temperature during 30 minutes and then pourred into water. After extraction with CH₂Cl₂, the organic phase was washed with water, dried over Na₂SO₄, and evaporated under reduced pressure. The title compound was obtained as a yellow solid (10.1 g).

m.p.: 121-123° C.

Intermediate 4

1-(3-Cyano-benzyl)-4-(5-nitro-piridin-2-yl)-piperazine

To a stirred solution of 5-nitro-2-piperazinyl-pyridine (10.1 g) and potassium carbonate (20.29 g) in acetone (500 mL) was added portionwise 3-cyano-benzyl bromide (9.6 g) and the mixture was heated under reflux during 2 hours. The salts were removed by filtration, washed with acetone and the filtrate was evaporated to dryness. The residue was taken in CH₂Cl₂, and the solution washed with water, dried over Na₂SO₄, filtered and evaporated under reduced pressure to leave an oil which crystallized by trituration with diisopropyl oxyde. The title compound was obtained as a yellow solid (14.4 g).

m.p.: 103-105° C.

Intermediate 5

1-Benzyl-4-(5-nitro-piridin-2-yl)-piperazine

To a stirred solution of 1-benzyl-piperazine (30 g) and triethylamine (20 mL) in THF (500 mL) was added 2-chloro-5-nitro-pyridine (30 g) and the mixture was heated under reflux during 2 hours and then evaporated to dryness. The residue was taken in water, and the precipitate was filtered and dried. After crystallisation from acetonitrile, the title compound was obtained as red crystals. (54 g).

m.p.: 124-126° C.

Intermediate 6

1-(terbutyloxycarbonyl)-4-(5-nitro-piridin-2-yl)-piperazine

To a stirred solution of 2-bromo-5-nitro-pyridine (2.9 g) and N-terbutyloxycarbonyl-piperazine (3.2 g) in DMF (100 mL) was added potassium carbonate (1.98 g). The mixture was heated at 80° C. during 1 hour and then concentrated. The residue was taken in CH₂Cl₂ and the organic phase was washed with water, dried over Na₂SO₄, filtered and evaporated under reduced pressure. The titled compound was obtained as a yellow solid (4.4 g).

m.p.: 169° C.

Intermediate 7

1-Benzyl-4-(5-amino-piridin-2-yl)-piperazine

To a stirred solution of 1-benzyl-4-(5-nitro-piridin-2-yl)-piperazine (54 g) in EtOH (300 mL) and THF (300 mL) was added portionwise SnCl₂.2H₂O (163 g) and the mixture was heated under reflux during 1.5 hour. After evaporation of the solvant, the residue was taken in water, basified with NaOH at pH 14 and extracted with CH₂Cl₂. The organic phase was then washed with water, dried over Na₂SO₄ and evaporated. The residue was triturated with diisopropyl oxyde and the solid was filtered and dried. The title compound was obtained as a dark red solid.

MS: m/z 269 (M+1).

Similarly Prepared Were:

Intermediate 8

1-(3-Cyano-benzyl)-4-(5-amino-piridin-2-yl)-piperazine as Brown Crystals (4.7 g),

m.p.: 119-121° C.

from 1-(3-cyano-benzyl)-4-(5-nitro-piridin-2-yl)-piperazine (14.4 g).

Intermediate 9

1-Benzyl-4-(5-amino-pyrimydin-2-yl)-piperazine as a Red Oil (1.5 g),

MS: m/z 270 (M+1)

from 1-benzyl-4-(5-nitro-pyrimydin-2-yl)-piperazine (2 g).

Intermediate 10

1-(terbutyloxycarbonyl)-4-(5-amino-piridin-2-yl)-piperazine

A solution of 1-(terbutyloxycarbonyl)-4-(5-nitro-piridin-2-yl)-piperazine (4.4 g) in EtOH (150 mL) containing Pd/C (0.5 g) was hydrogenated at room temperature during 3 hours. The catalyst was filtered off and the filtrate was evaporated under reduced pressure. The titled compound was obtained as a brown oil (3.9 g).

MS: m/z 279 (M+1).

Intermediate 11

4′-Isopropyl-6-methyl-biphenyl-2-carboxylic acid [2-(4-benzyl-piperazin-1-yl)-pyridin-5-yl]-amide

To a stirred solution of 1-benzyl-4-(5-amino-piridin-2-yl)-piperazine (2.68 g), 4′-isopropyl-6-methyl-biphenyl-2-carboxylic acid (2.54 g), HOBT (1.49 g) and triethylamine (1.6 mL) in CH₂Cl₂ (50 mL) was added EDCl (2.1 g) and the mixture was heated at 40° C. overnight. The mixture was diluted with CH₂Cl₂, and the organic solution was washed with water, then with a saturated solution of NaHCO₃, then with a saturated solution of NaCl and dried over Na₂SO₄. After filtration and evaporation of the filtrate, the residue was purified by flash chromatography eluting with AcOEt/CH₂Cl₂ (50/50) to give the title compound as a white powder (2.9 g).

m.p.: 138-140° C.

Similarly Prapared Were:

Intermediate 12

6-Methyl-4′-trifluoromethyl-biphenyl-2-carboxylic acid [2-(4-benzyl-piperazin-1-yl)-pyridin-5-yl]-amide as White Powder (3.9 g),

m.p.: 100° C.

from 1-benzyl-4-(5-amino-piridin-2yl)-piperazine (2.68 g) and 6-methyl4′-trifluoromethyl-biphenyl-2-carboxylic acid (2.8 g).

Intermediate 13

4′-Isopropyl-6-methoxy-biphenyl-2-carboxylic acid [2-(4-benzyl-piperazin-1-yl)-pyridin-5-yl]-amide as a White Powder (4 g),

m.p.: 158-160° C.

from 1-benzyl-4-(5-amino-piridin-2-yl)-piperazine (2.68 g) and 4′-isopropyl-6-methoxy-biphenyl-2-carboxylic acid (2.7 g).

Intermediate 14

4′-Trifluoromethyl-biphenyl-2-carboxylic acid [2-(4-benzyl-piperazin-1-yl)-pyridin-5-yl]-amide as White Powder (0.85 g),

MS: m/z 517 (M+1)

from 1-benzyl-4-(5-amino-piridin-2-yl)-piperazine (0.81 g) and 4′-trifluoromethyl-biphenyl-2-carboxylic acid (0.8 g).

Intermediate 15

4′-Trifluoromethyl-biphenyl-2-carboxylic acid [2-(4-benzyl-piperazin-1-yl)-pyrimydin-5-yl]-amide as a White Powder (1.4 g),

m.p.: 202-204° C.

from 1-benzyl4-(5-amino-pyrimydin-2-yl)-piperazine (1.5 g) and 4′-trifluoro-methyl-biphenyl-2-carboxylic acid (1.49 g).

Intermediate 16

4′,6-Diisopropyl-biphenyl-2-carboxylic acid [2-(4-terbutyloxycarbonyl-piperazin-1-yl)-pyridin-5-yl]-amide as Pink Crystals (6.69 g),

m.p.: 171° C.

from 1-terbutyloxycarbonyl-4-(5-amino-piridin-2-yl)-piperazine (3.9 g) and 4′,6-diisopropyl-biphenyl-2-carboxylic acid (3.96 g).

Intermediate 17

4′-Isopropyl-6-methyl-biphenyl-2-carboxylic acid [2-(piperazinyl)-pyridin-5-yl]-amide

A solution of 4′-isopropyl-6-methyl-biphenyl-2-carboxylic acid [2-(4-benzyl-piperazin-1-yl)-pyridin-5-yl]-amide (2.9 g) in EtOH (200 mL) and CH₂Cl₂ (10 mL) containing Pd/C, was hydrogenated at room temperature. After 24 hours, the catalyst was removed by filtration and the filtrate was evaporated under reduced pressure. The titled compound was obtained as a white powder (1 g).

m.p.: 140° C.

Similarly Prepared Were:

Intermediate 18

6-Methyl-4′-trifluoromethyl-biphenyl-2-carboxylic acid [2-(piperazinyl)-pyridin-5-yl]-amide as Cream Powder (2.7 g),

m.p.: 150° C.

from 6-methyl-4′-trifluoromethyl-biphenyl-2-carboxylic acid [2-(4-benzyl-piperazin-1-yl)-pyridin-5-yl]-amide (3.9 g).

Intermediate 19

4′-Isopropyl-6-methoxy-biphenyl-2-carboxylic acid [2-(piperazinyl)-pyridin-5-yl]-amide as Cream Powder (3 g),

m.p.: 160° C.

from 4′-isopropyl-6-methoxy-biphenyl-2-carboxylic acid [2-(4-benzyl-piperazin-1-yl)-pyridin-5-yl]-amide (4 g).

Intermediate 20

4′-Trifluoromethyl-biphenyl-2-carboxylic acid [2-(piperazinyl)-pyridin-5-yl]-amide as Colorless Oil (0.6 g),

MS: m/z 427 (M+1)

from 4′-trifluoromethyl-biphenyl-2-carboxylic acid [2-(4-benzyl-piperazin-1-yl)-pyridin-5-yl]-amide (0.85 g).

Intermediate 21

4′-Trifluoromethyl-biphenyl-2-carboxylic acid [2-(piperazinyl)-pyrimydin-5-yl]-amide as a White Powder (1 g),

MS: m/z 428 (M+1)

from 4′-trifluoromethyl-biphenyl-2-carboxylic acid [2-(4-benzyl-piperazin-1-yl)-pyrimydin-5-yl]-amide (1.4 g).

Intermediate 22

4′,6-Disopropyl-biphenyl-2-carboxylic acid [2-(piperazinyl)-pyridin-5-yl]-amide

To a stirred solution of 4′,6-diisopropyl-biphenyl-2-carboxylic acid (2-(4-terbutyloxycarbonyl-piperazin-1-yl)-pyridin-5-yl]-amide (6.69 g) in CH₂Cl₂ (100 mL) was added dropwise trifluoroacetic acid (9.75 mL). The mixture was stirred at room temperature during 4 hours and then pourred into a solution of NaOH 1N. After extraction with CH₂Cl₂, the organic phase was dried over Na₂SO₄ filtered and evaporated under reduced pressure. The title compound was obtained as a light brown solid (5.4 g).

MS: m/z 443 (M+1).

### EXAMPLE 1

4′-Isopropyl-6-methyl-biphenyl-2-carboxylic acid [2-(4-carbamoylmethyl-piperazin-1-yl)-pyridin-5-yl]-amide

To a solution of 4′-isopropyl-6-methyl-biphenyl-2-carboxylic acid [2-(piperazinyl)-pyridin-5-yl]-amide (300 mg) in THF (20 mL) containing triethylamine (0.12 mL) was added 2-bromo-acetamide (120 mg) and the mixture was heated under reflux during 4 hours and then concentrated. The residue was treated with water, extracted with CH₂Cl₂. The organic phase was dried over Na₂SO₄ and evaporated under reduced pressure. The title compound was obtained as a white powder (130 mg).

m.p.: 200-202° C.

Analysis: C28H33N5O2

Calc: C,71.31; H,7.05; N,14.85;

Found: C,71.51; H,6.99; N,14.32%.

Similarly Prepared Were:

### EXAMPLE 2

6-Methyl-4′-trifluoromethyl-biphenyl-2-carboxylic acid [2-(4-carbamoylmethyl-piperazin-1-yl)-pyridin-5-yl]-amide as a White Powder (210 mg),

m.p.: 180-181° C.

MS: m/z 498 (M+1)

from 6-methyl-4′-trifluoromethyl-biphenyl-2-carboxylic acid [2-(piperazinyl)-pyridin-5-yl]-amide (400 mg).

### EXAMPLE 3

4′-Isopropyl-6-methoxy-biphenyl-2-carboxylic acid [2-(4-carbamoylmethyl-piperazin-1-yl)-pyridin-5-yl]-amide as a White Powder (170 mg),

m.p.: 210-212° C.

MS: m/z 488 (M+1)

from 4′-isopropyl-6-methoxy-biphenyl-2-carboxylic acid [2-(piperazinyl)-pyridin-5-yl]-amide (400 mg).

### EXAMPLE 4

4′,6-Diisopropyl-biphenyl-2-carboxylic acid [2-(4-carbamoylmethyl-piperazin-1-yl)-pyridin-5-yl]-amide as Light Yellow Crystals (185 mg),

m.p.: 169° C.

MS: m/z 500 (M+1)

from 4′,6-diisopropyl-biphenyl-2-carboxylic acid [2-(piperazinyl)-pyridin-5-yl]-amide (250 mg).

### EXAMPLE 5

4′-Isopropyl-6-methyl-biphenyl-2-carboxylic acid [2-(4-(1H-pyrrol-2-ylmethyl)piperazin-1-yl)-pyridin-5-yl]-amide

To a solution of 4′-isopropyl-6-methyl-biphenyl-2-carboxylic acid [2-(piperazinyl)-pyridin-5-yl]-amide (300 mg) in CH₂Cl₂ (30 mL) was added 1H-pyrrole-2-carboxaldehyde (76 mg) and then sodium triacetoxy borohydride (170 mg). The mixture was stirred at room temperature during 24 hours. The solution was then washed with a solution of NaOH 0.5N, with brine, dried over Na₂SO₄, filtered and evaporated under reduced pressure. The residue was purified by flash chromatography eluting with CH₂Cl₂/MeOH (95/5) and the solid obtained was crystallized from pentane to give the title compound as light brown crystals (150 mg).

m.p.: 130° C.

MS: m/z 494 (M+1)

Similarly prepared were:

### EXAMPLE 6

6-Methyl4′-trifluoromethyl-biphenyl-2-carboxylic acid [2-(4-(1H-pyrrol-2-ylmethyl)piperazin-1-yl)-pyridin-5-yl]-amide as a White Powder (190 mg),

m.p.: 142-144° C.

MS: m/z 520 (M+1)

from 6-methyl-4′-trifluoromethyl-biphenyl-2-carboxylic acid [2-(piperazinyl)-pyridin-5-yl]-amide (300 mg).

### EXAMPLE 7

4′-Isopropyl-6-methoxy-biphenyl-2-carboxylic acid [2-(4-(1H-pyrrol-2-ylmethyl)piperazin-1-yl)-pyridin-5-yl]-amide as a White Powder (220 mg),

m.p.: 197-199° C.

Analysis: C31H35N5O2

Calc: C 73.06 H 6.92 N 13.74

Found: C 72.58 H 6.64 N 13.55

from 4′-isopropyl-6-methoxy-biphenyl-2-carboxylic acid [2-(piperazinyl)-pyridin-5-yl]-amide (400 mg).

### EXAMPLE 8

4′-6-Diisopropyl-biphenyl-2-carboxylic acid [2-(4-(1H-pyrrol-2-ylmethyl)piperazin-1-yl)-pyridin-5-yl]-amide as Cream Crystals (120 mg),

m.p.: 135° C.

MS: m/z 522 (M+1)

from 4′-6-diisopropyl-biphenyl-2-carboxylic acid [2-(piperazinyl)-pyridin-5-yl]-amide (200 mg).

### EXAMPLE 9

4′-Trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide as a White Powder (400 mg),

m.p.: 192-194° C.

Analysis: C33H29F3N6O2

Calc: C,66.21; H,4.88; N,14.04;

Found: C,66.30; H,4.63; N,13.60%.

from 4′-trifluoromethyl-biphenyl-2-carboxylic acid [2-(piperazinyl)-pyridin-5-yl]-amide (300 mg) and 3-(3-methyl-[1,2,4]oxadiazol-5-yl)-benzaldehyde (141 mg).

### EXAMPLE 10

4′-Isopropyl-6-methyl-biphenyl-2-carboxylic acid [2-(4-(3-(3-methyl-[1,2,4]oxadiazol-5-yl)-benzyl)-piperazin-1-yl)-pyridin-5-yl]-amide as a White Powder (200 mg),

m.p.: 150-152° C.

MS: m/z 587 (M+1)

from 4′-isopropyl-6-methyl-biphenyl-2-carboxylic acid [2-(piperazinyl)-pyridin-5-yl]-amide (300 mg) and 3-(3-methyl-[1,2,4]oxadiazol-5-yl)-benzaldehyde (150 mg).

### EXAMPLE 11

6-Methyl-4′-trifluoromethyl-biphenyl-2-carboxylic acid [2-(4-(3-(3-methyl-[1,2,4oxadiazol-5-yl)-benzyl)-piperazin-1-yl)-pyridin-5-yl]-amide as a White Powder (200 mg),

m.p.: 170-172° C.

MS: m/z 613 (M+1)

from 6-methyl-4′-trifluoromethyl-biphenyl-2-carboxylic acid [2-(piperazinyl)-pyridin-5-yl]-amide (300 mg) and 3-(3-methyl-[1,2,4]oxadiazol-5-yl)-benzaldehyde (141 mg).

### EXAMPLE 12

4′-Isopropyl-6-methoxy-biphenyl-2-carboxylic acid [2-(4-(3-(3-methyl-[1,2,4]oxadiazol-5-yl)-benzyl)-piperazin-1-yl)-pyridin-5-yl]-amide as a White Powder (100 mg),

m.p.: 136-138° C.

MS/m/z 603 (M+1)

from 4′-isopropyl-6-methoxy-biphenyl-2-carboxylic acid [2-(piperazinyl)-pyridin-5-yl]-amide (300 mg) and 3-(3-methyl-[1,2,4]oxadiazol-5-yl)-benzaldehyde (145 mg).

### EXAMPLE 13

4′,6-Diisopropyl-biphenyl-2-carboxylic acid [2-(4-(3-(3-methyl-[1,2,4]oxadiazol-5-yl)-benzyl)-piperazin-1-yl)-pyridin-5-yl]-amide as White Crystals (95 mg),

m.p.: 125° C.

MS: m/z615 (M+1)

from 4′,6-diisopropyl-biphenyl-2-carboxylic acid [2-(piperazinyl)-pyridin-5-yl]-amide (250 mg) and 3-(3-methyl-[1,2,4]oxadiazol-5-yl)-benzaldehyde (111 mg).

### EXAMPLE 14

4′-Trifluoromethyl-biphenyl-2-carboxylic acid [2-(4-(furan-2-ylmethyl)piperazin-1-yl)-pyridin-5-yl]-amide as Light Yellow Crystals (169 mg),

m.p.: 149° C.

Analysis: C28H25F3N4O2

Calc: C,66.39; H,4.97; N,11.06;

Found: C,66.26; H,5.43; N,11.00%.

from 4-trifluoromethyl-biphenyl-2-carboxylic acid [2-(piperazinyl)-pyridin-5-yl]-amide (300 mg) and furfuraldehyde (68 mg).

### EXAMPLE 15

4′,6-Diisopropyl-biphenyl-2-carboxylic acid [2-(4-(3-cyano-benzyl)-piperazin-1-yl)-pyridin-5-yl]-amide as Ecru Crystals (130 mg),

m.p.: 151° C.

MS: m/z 558 (M+1)

from 4′,6-diisopropyl-biphenyl-2-carboxylic acid [2-(piperazinyl)-pyridin-5-yl]-amide (250 mg) and 3-cyanobenzaldehyde (78 mg).

### EXAMPLE 16

4′-Trifluoromethyl-biphenyl-2-carboxylic acid [2-(4-(3-cyano-benzyl)-piperazin-1-yl)-pyrimydin-5-yl]-amide as a White Powder (50 mg),

m.p. : 130-132° C.

MS: m/z 543 (M+1)

from 4′-trifluoromethyl-biphenyl-2-carboxylic acid [2-(piperazinyl)-pyrimydin-5-yl]-amide (250 mg) and 3-cyanobenzaldehyde (78 mg).

### EXAMPLE 17

4′-Trifluoromethyl-biphenyl-2-carboxylic acid [2-(4-(3-cyano-benzyl)-piperazin-1-yl)-pyridin-5-yl]-amide

To a stirred solution of 4′-trifluoromethyl-biphenyl-2-carboxylic acid [2-(piperazinyl)-pyridin-5-yl]-amide (300 mg) and sodium hydrogenocarbonate (65 mg) in acetone (30 mL) was added 3-cyano-benzyl bromide (145 mg). The mixture was heated under reflux during 2 hours and then pourred into water. After extraction with CH₂Cl₂, the organic phase was dried over Na₂SO₄ and evaporated under reduced pressure. The residue was purified by flash chromatography eluting with CH₂Cl₂/MeOH (98/2 then 9515), and crystallized from diisopropyl oxyde. The title compound was obtained as ecru crystals (263 mg).

m.p. : 168° C.

MS: m/z 542 (M+1);

### EXAMPLE 18

4′-Isopropyl-6-methoxy-biphenyl-2-carboxylic acid [2-(4-(3-cyano-benzyl)-piperazin-1-yl)-pyridin-5-yl]-amide

To a stirred solution of 1-(3-cyano-benzyl)-4-(5-amino-piridin-2-yl)-piperazine (400 mg), 4′-isopropyl-6-methoxy-biphenyl-2-carboxylic acid (365 mg), HOBT (220 mg) and triethylamine (0.228 mL) in CH₂Cl₂ (20 mL) was added EDCl (313 mg) and the mixture was stirred at room temperature overnight. The mixture was diluted with CH₂Cl₂, and the organic solution was washed with water, then with a saturated solution of NaHCO₃, then with a saturated solution of NaCl and dried over Na₂SO₄. After filtration and evaporation of the filtrate, the residue was purified by flash chromatography eluting with AcOEt/CH₂Cl₂ (50/50). After crystallization from CH₃CN, the title compound was obtained as white crystals (360 mg).

m.p.: 194-196° C.

MS: m/z 546 (M+1).

Similarly prepared were:

### EXAMPLE 19

4′-Isopropyl-6-methyl-biphenyl-2-carboxylic acid [2-(4-(3-cyano-benzyl)-piperazin-1-yl)-pyridin-5-yl]-amide as a White Powder (230 mg),

m.p.: 154-156° C.

Analysis: C34H35N5O1

Calc: C,77.10; H,6.66; N,13.22;

Found: C,76.63; H,6.26; N,13.17%.

from 1-(3-cyano-benzyl)-4-(5-amino-pyridin-2-yl)-piperazine (400 mg), 4′-isopropyl-6-methyl-biphenyl-2-carboxylic acid (340 mg).

### EXAMPLE 20

6-Methyl-4′-trifluoromethyl-biphenyl-2-carboxylic acid [2-(4-(3-cyano-benzyl)-piperazin-1-yl)-pyridin-5-yl]-amide as a White Powder (180 mg),

m.p.: 164-166° C.

MS: m/z 556 (M+1)

from 1-(3-cyano-benzyl)-4-(5-amino-piridin-2-yl)-piperazine (400 mg), 6-methyl-4′-trifluoromethyl-biphenyl-2-carboxylic acid (380 mg).

### EXAMPLE 21

4′-Isopropyl-5-methyl-biphenyl-2-carboxylic acid [2-(4-(3-cyano-benzyl)-piperazin-1-yl)-pyridin-5-yl]-amide as White Crystals (120 mg),

m.p.: 161-163° C.

Analysis: C34H35N5O1

Calc: C,77.10; H,6.66; N,13.22;

Found: C,76.74; H,6.47; N,13.07%.

from 1-(3-cyano-benzyl)-4-(5-amino-piridin-2-yl)-piperazine (150 mg), 4′-isopropyl-5-methyl-biphenyl-2-carboxylic acid (127 mg).

### EXAMPLE 22

5-Methyl-4′-trifluoromethyl-biphenyl-2-carboxylic acid [2-(4-(3-cyano-benzyl)-piperazin-1-yl)-pyridin-5-yl]-amide as White Crystals (260 mg),

m.p.: 158-160° C.

MS: m/z 556 (M+1)

from 1-(3-cyano-benzyl)-4-(5-amino-piridin-2-yl)-piperazine (400 mg), 6-methyl-4′-trifluoromethyl-biphenyl-2-carboxylic acid (380 mg).

EXAMPLE 23

4′,6-Diisopropyl-biphenyl-2-carboxylic acid-[2-(4-propyl-piperazin-1-yl)-pyridin-5-yl]-amide

To a stirred solution of 4′,6-diisopropyl-biphenyl-2-carboxylic acid [2-(piperazinyl)-pyridin-5-yl]-amide (442 mg) and cesium carbonate (391 mg) in acetone (30 mL) was added 1-bromopropane (148 mg). The mixture was heated under reflux overnight and then pourred into water. After extraction with CH₂Cl₂, the organic phase was dried over Na₂SO₄ and evaporated under reduced pressure. The residue was purified by chromatography on silicagel eluting with CH₂Cl₂/MeOH (95/5). After trituration with pentane, the title compound was obtained a cream powder (210 mg).

m.p.: 126-128° C.

MS: m/z 485 (M+1).

Similarly prepared were:

### EXAMPLE 24

4′,6-Diisopropyl-biphenyl-2-carboxylic acid-[2-(4-methyl-piperazin-1-yl)-pyridin-5-yl]-amide as a White Powder (500 mg),

m.p.: 152-154° C.

MS: m/z 457 (M+1)

from 4′,6-diisopropyl-biphenyl-2-carboxylic acid [2-(piperazinyl)-pyridin-5-yl]-amide

(550 mg).

### EXAMPLE 25

4′,6-Diisopropyl-biphenyl-2-carboxylic acid-[2-(4-(propen-2-yl)-piperazin-1-yl)-pyridin-5-yl]-amide as a yellow powder (138 mg),

m.p.: 124° C.

MS: m/z 483 (M+1)

from 4′,6-diisopropyl-biphenyl-2-carboxylic acid [2-(piperazinyl)-pyridin-5-yl]-amide

(200 mg).

### EXAMPLE 26

4′,6-Diisopropyl-biphenyl-2-carboxylic acid-[2-(4-(isopropyl)-piperazin-1-yl)-pyridin-5-yl]-amide as a White Powder (123 mg),

m.p.: 136° C.

MS: m/z 485 (M+1)

from 4′,6-diisopropyl-biphenyl-2-carboxylic acid [2-(piperazinyl)-pyridin-5-yl]-amide

(200 mg).

Biological Assay

ApoB-100 Assay

Primary human hepatocytes were seeded at 50 000 cells/well in 96 well plates. After an overnight adhesion phase, cells were incubated with compounds for 8 hours in RPMI medium containing 1% FCS, 4 μg/ml insulin, 100 nM dexamethasone and 50 μCi/ml ³⁵S-methionine. Compounds were dissolved in DMSO and tested onto cells from 1 μM to 1.6 nM. Production of radiolabeled apoB-100 and apoA-1 (used as a selectivity control) was quantified by analysis of supernatants using SDS PAGE and exposure of gels onto PhosphorImager screens. Inhibition of apoB-100 and apoA-1 secretion by compounds was calculated taking untreated cells as controls, and IC₅₀ of each compound was determined on both apoproteins.

MTP Assay

The human MTP activity assay was established using SPA technology. Donor liposomes were prepared with 3H-triolein and phosphatidylcholine, while acceptor liposomes contained biotinylated phosphatidylethanolamine and phosphatidylcholine. The MTP-mediated 3H-triolein transfer onto acceptor liposomes was allowed by a 25 min incubation at 37° C., and quantified by the addition of streptavidin-SPA beads. Results for a range of compounds are shown below.

| Example | MTP (nM) |
|---|---|
| 1 | 0.3 |
| 5 | 0.1 |
| 15 | 0.16 |
| 25 | <0.1 |

Tablet Compositions

The following compositions A and B can be prepared by wet granulation of ingredients (a) to (c) and (a) to (d) with a solution of povidone, followed by addition of the magnesium stearate and compression.

| | | |
|---|---|---|
| Composition A | mg/tablet | mg/tablet |

| | | | |
|---|---|---|---|
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |

| | | |
|---|---|---|
| Composition B | mg/tablet | mg/tablet |

| | | | |
|---|---|---|---|
| (a) | Active ingredient | 250 | 250 |
| (b) | Lactose 150 | 150 | — |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |

| Composition C | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in composition E is of the direct compression type.

| Composition D | mg/tablet |
|---|---|
| Active ingredient | 250 |
| Magnesium Stearate | 4 |
| Pregelatinised Starch NF15 | 146 |
| | 400 |

| Composition E | mg/tablet |
|---|---|
| Active ingredient | 250 |
| Magnesium Stearate | 5 |
| Lactose | 145 |
| Avicel | 100 |
| | 500 |

| Composition F | | |
|---|---|---|
| (Controlled release composition) | | mg/tablet |
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |
| | | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl- cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl- cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

| Composition B | | mg/capsule |
|---|---|---|
| (a) | Active ingredient | 250 |
| (b) | Lactose B.P. | 143 |
| (c) | Sodium Starch Glycollate | 25 |
| (d) | Magnesium Stearate | 2 |
| | | 420 |

| Composition C | | mg/capsule |
|---|---|---|
| (a) | Active ingredient | 250 |
| (b) | Macrogol 4000 BP | 350 |
| | | 600 |

Capsules can be prepared by melting the Macrogol 4000 BP, dispersing the active ingredient in the melt and filling two-part hard gelatin capsules therewith.

| Composition D | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

| Composition E | | |
|---|---|---|
| (Controlled release capsule) | | mg/capsule |
| (a) | Active ingredient | 250 |
| (b) | Microcrystalline Cellulose | 125 |
| (c) | Lactose BP | 125 |
| (d) | Ethyl Cellulose | 13 |
| | | 513 |

The controlled release capsule composition can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)
mg/capsule

| | | |
|---|---|---|
| (a) | Active ingredient | 250 |
| (b) | Microcrystalline Cellulose | 125 |
| (c) | Lactose BP | 125 |
| (d) | Cellulose Acetate Phthalate | 50 |
| (e) | Diethyl Phthalate | 5 |
| | | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(iii) Intravenous injection composition

| | |
|---|---|
| Active ingredient | 0.200 g |
| Sterile, pyrogen-free phosphate buffer (pH 9.0) to | 10 ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | |
|---|---|
| Active ingredient | 0.20 g |
| Benzyl Alcohol | 0.10 g |
| Glycofurol 75 | 1.45 g |
| Water for injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

| (vi) Suppository composition | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

| (vii) Pessary composition | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

| (viii) Transdermal composition | | |
|---|---|---|
| Active ingredient | 200 | mg |
| Alcohol USP | 0.1 | ml |
| Hydroxyethyl cellulose | | |

The active ingredient and alcohol USP are gelled with hydroxyethyl cellulose and packed in a transdermal device with a surface area of 10 cm².

1. A compound of formula (I) 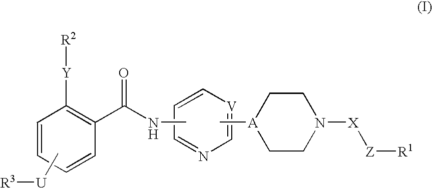
wherein:
A represents N or CH;
U represents a direct link, —C₁₋₄alkylene- or —C₀₋₄alkylene-oxy-C₀₋₄alkylene-;
V represents N or CH;
X is selected from the following groups:
(i) —C₁₋₆alkylene-, optionally containing one or two double bonds and optionally substituted by one or more hydroxy, C₁₋₆ alkyl, C₁₋₆alkoxy, C₁₋₆acyl or C₁₋₆acyloxy groups,
(ii) oxo, sulfonyl, thioxo,
(iii) —C₁₋₆alkylenecarbonyl-, —C₁₋₆alkylenesulfonyl-, —C₁₋₆alkylenethioxo-,
(iv) —C₂₋₆alkyleneoxy-, —C₂₋₆alkylenethio-, —C₂₋₆alkylene(N—H or N—C₁₋₆alkyl)amino-,
(v) —C₁₋₆alkylenecarboxy-, —C₁₋₆alkylenethioamido-, —C₁₋₆alkylene(N—H or N—C₁₋₆alkyl)carboxamido-, and
(vi) —C₂₋₆alkyleneoxycarbonyl-, —C₂₋₆alkylenethiocarbonyl-, —C₂₋₆alkylene(N—H or N—C₁₋₆alkyl)aminocarbonyl-;
Z represents a direct link or —C₁₋₆alkylene-, optionally containing one double bond and optionally substituted by one or more hydroxy, C₁₋₆alkyl, C₁₋₆ alkoxy,
C₁₋₆ acyl or C₁₋₆ acyloxy groups;
R¹ is selected from the following groups:
(i) hydrogen, C₁₋₃perfluoroalkyl,
(ii) C₆₋₁₀aryl, C₃₋₈cycloalkyl and fused benz derivatives thereof, C₇₋₁₀polycycloalkyl, C₄₋₈cycloalkenyl, C₇₋₁₀polycycloalkenyl,
(iii) a heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, and
(iv) where either X is C₁₋₆alkylene and Z is a direct link, or Z is C₁₋₆alkylene, R¹ additionally may represent a halogen, cyano, nitro or C₁₋₆acyl group;
wherein, when R¹ contains one or more rings, said rings may each independently bear 0 to 4 substituents independently selected from:
(i) halogen, hydroxy, cyano, nitro, formyl, C₁₋₆alkylsulfonylamino,
(ii) C₁₋₆alkyl, C₃₋₈cycloalkyl, C₁₋₃perfluoroalkyl,
(iii) C₁₋₆alkoxy, methylenedioxy, C₁₋₃perfluoroalkoxy, C₁₋₆alkylthio,
(iv) amino, C₁₋₆alkylamino, di-C₁₋₆alkylamino,
(v) phenyl, phenoxy, phenylthio, halophenylthio, benzyl, benzyloxy,
(vi) hydroxycarbonyl, C₁₋₆alkoxycarbonyl,
(vii) aminocarbonyl, C₁₋₆alkylaminocarbonyl, di-C₁₋₆alkylaminocarbonyl, di-C₁₋₆alkylaminocarbonylC₁₋₆alkoxy, C₁₋₃perfluoroalkylaminocarbonyl,
(viii) C₁₋₆acyl, C₁₋₆acyloxy, C₁₋₆acyloxyC₁₋₆alkyl, C₁₋₆acylamino, and
(ix) an aromatic heterocyclyl consisting of monocyclic radicals, wherein said radicals contain 5-6 ring atoms, wherein said radicals contain a total of from 1-4 ring heteroatoms independently selected from oxygen, nitrogen and sulfur, and where each of the said heterocyclyl groups is optionally substituted by one or more groups independently selected from halogen, C₁₋₄alkyl, C₁₋₄alkoxy, C₁₋₃ perfuoroalkyl and C₁₋₃perfuoroalkoxy;
Y represents a direct or oxy link, —C₁₋₆alkylene-, -oxyC₁₋₆alkylene- or a heterocyclyl consisting of monocyclic radicals, wherein said radicals contain 5 ring atoms, and wherein said radicals contain a total of from 1-4 ring heteroatoms independently selected from oxygen, nitrogen and sulfur and wherein the ring may be independently saturated, partially unsaturated, or aromatic;
R² represents phenyl, C₃₋₈cycloalkyl, or a heterocyclyl consisting of monocyclic radicals, wherein said radicals contain a total of from 5-6 ring atoms, wherein said radicals contain a total of from 1-4 ring heteroatoms independently selected from oxygen, nitrogen and sulfur, wherein the ring may be independently saturated, partially unsaturated, or aromatic, and where each R² is optionally substituted by one or more groups independently selected from halogen, C₁₋₄alkyl, C₁₋₄alkoxy, C₃₋₈cycloalkyl, C₁₋₃perfuoroalkyl, C₁₋₃perfuoroalkoxy, hydroxycarbonyl, C₁₋₆alkoxycarbonyl, cyano, nitro and C₁₋₄alkylaminosulfonyl;
R³ is selected from the following groups:
(i) hydrogen or C₁₋₃perfluoroalkyl,
(ii) phenyl or a heterocyclyl consisting of monocyclic radicals, wherein said radicals contain a total of 5-6 ring atoms, wherein said radicals contain a total of from 1-4 ring heteroatoms selecetd from oxygen, nitrogen or sulfur, and wherein the ring may be saturated, partially unsaturated or aromatic,
(iii) cyano, hydroxycarbonyl, C₁₋₆alkoxycarbonyl, aminocarbonyl, C₁₋₆alkylaminocarbonyl or C₁₋₆dialkylaminocarbonyl, with the proviso that U may not represent —C₀₋₄alkylene-oxy-,
(iv) halogen, amino, C₁₋₆alkylamino or C₁₋₆dialkylamino, with the proviso that U may not represent —C₀₋₄alkylene-oxy-C₀₋₁alkylene,
wherein, when R³ contains one or more rings, said rings may each independently bear 0 to 4 substituents independently selected from C₁₋₆alkyl, C₁₋₆alkoxy, hydroxy and halogen;
or a physiologically acceptable salt, solvate or derivative thereof.

2. A compound according to claim 1 where A represents N and V represents CH.

3. A compound according to claim 1 or 2 where X is a methylene, propylene, prop-2-enylene or methylene(N—H)carboxamido.

4. A compound according to any one of claims 1-3 where Z is a direct link or —C₁₋₆alkylene-.

5. A compound according to any one of claims 1-4 where R¹ is selected from hydrogen, substituted phenyl, where substitution is effected by cyano or a methyl substituted [1,2,4]-oxadiazol-5-yl group, or a pyrrolyl or furanyl group.

6. A compound according to any one of claims 1-5 where —X-Z-R¹ is methyl, n-propyl, prop-2-enyl, aminocarbonylmethyl, pyrrolylmethyl or phenylmethyl substituted by 3-cyano or 3-(3-methyl-[1,2,4]-oxadiazol-5-yl).

7. A compound according to any one of claims 1-6 where Y is suitably a direct link, a 2,5-substituted oxazolyl group, or —(CH₂)ₙ—O—, where n is an integer from 0-3.

8. A compound according to any one of claims 1-7 where R² is a phenyl group substituted by a trifluoromethyl group, most preferably in the 4-position, or R² is a phenyl group substituted by an isopropyl group, most preferably in the 4-position.

9. A compound according to any one of claims 1-8 where U-R³ is hydrogen, halogen, C₁₋₄ alkyl, C₁₋₄alkoxy, C₁₋₃perfluoroalkyl, C₁₋₆dialkylamino or methylenedialkylamino.

10. A compound according t claim 1 which is represented by a compound of formula (Ic) 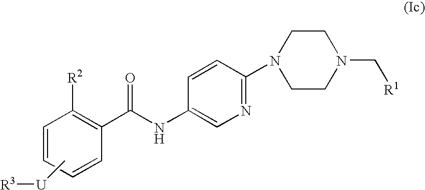
wherein
U—R³ is suitably hydrogen, halogen, C₁₋₄ alkyl, C₁₋₄alkoxy or C₁₋₃perfluoroalkyl;
R¹ represents phenyl optionally substitued by one or two groups independently selected from C₁₋₆ alkyl, cyano, halogen, C₁₋₆alkoxy, trifluoromethyl, hydroxycarbonyl and C₁₋₆alkoxycarbonyl;
R² represents phenyl substituted in the 4-position by a halogen, trifluoromethyl, C₁₋₄alkyl or C₁₋₄alkoxy group;
or a physiologically acceptable salt, solvate or derivative thereof.

11. A compound according to claim 1 which is selected from:
4′-isopropyl-6-methyl-biphenyl-2-carboxylic acid [2-(4-carbamoylmethyl-piperazin-1-yl)-pyridin-5-yl]-amide;
6-methyl-4′-trifluoromethyl-biphenyl-2-carboxylic acid [2-(4-carbamoylmethyl-piperazin-1-yl)-pyridin-5-yl]-amide;
4′-isopropyl-6-methoxy-biphenyl-2-carboxylic acid [2-(4-carbamoylmethyl-piperazin-1-yl)-pyridin-5-yl]-amide;
4′,6-diisopropyl-biphenyl-2-carboxylic acid [2-(4-carbamoylmethyl-piperazin-1-yl)-pyridin-5-yl]-amide;
4′-isopropyl-6-methyl-biphenyl-2-carboxylic acid [2-(4-(1H-pyrrol-2-ylmethyl)piperazin-1-yl)-pyridin-5-yl]-amide;
6-methyl-4′-trifluoromethyl-biphenyl-2-carboxylic acid [2-(4-(1H-pyrrol-2-ylmethyl)piperazin-1-yl)-pyridin-5-yl]-amide;
4′-isopropyl-6-methoxy-biphenyl-2-carboxylic acid [2-(4-(1H-pyrrol-2-ylmethyl)piperazin-1-yl)-pyridin-5-yl]-amide;
4′-6-diisopropyl-biphenyl-2-carboxylic acid [2-(4-(1H-pyrrol-2-ylmethyl)piperazin-1-yl)-pyridin-5-yl]-amide;
4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;
4′-isopropyl-6-methyl-biphenyl-2-carboxylic acid [2-(4-(3-(3-methyl-[1,2,4]oxadiazol-5-yl)-benzyl)-piperazin-1-yl)-pyridin-5-yl]-amide;
6-methyl-4′-trifluoromethyl-biphenyl-2-carboxylic acid [2-(4-(3-(3-methyl-[1,2,4]oxadiazol-5-yl)-benzyl)-piperazin-1-yl)-pyridin-5-yl]-amide;
4′-isopropyl-6-methoxy-biphenyl-2-carboxylic acid [2-(4-(3-(3-methyl-[1,2,4]oxadiazol-5-yl)-benzyl)-piperazin-1-yl)-pyridin-5-yl]-amide;
4′,6-diisopropyl-biphenyl-2-carboxylic acid [2-(4-(3-(3-methyl-[1,2,4]oxadiazol-5-yl)-benzyl)-piperazin-1-yl)-pyridin-5-yl]-amide;
4′-trifluoromethyl-biphenyl-2-carboxylic acid [2-(4-(furan-2-ylmethyl)piperazin-1-yl)-pyridin-5-yl]-amide;
4′,6-diisopropyl-biphenyl-2-carboxylic acid [2-(4-(3-cyano-benzyl)-piperazin-1-yl)-pyridin-5-yl]-amide;
4′-trifluoromethyl-biphenyl-2-carboxylic acid [2-(4-(3-cyano-benzyl)-piperazin-1-yl)-pyrimydin-5-yl]-amide;
4′-trifluoromethyl-biphenyl-2-carboxylic acid [2-(4-(3-cyano-benzyl)-piperazin-1-yl)-pyridin-5-yl]-amide;
4′-isopropyl-6-methoxy-biphenyl-2-carboxylic acid [2-(4-(3-cyano-benzyl)-piperazin-1-yl)-pyridin-5-yl]-amide;
4′-isopropyl-6-methyl-biphenyl-2-carboxylic acid [2-(4-(3-cyano-benzyl)-piperazin-1-yl)-pyridin-5-yl]-amide;
6-methyl-4′-trifluoromethyl-biphenyl-2-carboxylic acid [2-(4-(3-cyano-benzyl)-piperazin-1-yl)-pyridin-5-yl]-amide;
4′-isopropyl-5-methyl-biphenyl-2-carboxylic acid [2-(4-(3-cyano-benzyl)-piperazin-1-yl)-pyridin-5-yl]-amide;
5-methyl-4′-trifluoromethyl-biphenyl-2-carboxylic acid [2-(4-(3-cyano-benzyl)-piperazin-1-yl)-pyridin-5-yl]-amide;
4′,6-diisopropyl-biphenyl-2-carboxylic acid-[2-(4-propyl-piperazin-1-yl)-pyridin-5-yl]-amide;
4′,6-diisopropyl-biphenyl-2-carboxylic acid-[2-(4-methyl-piperazin-1-yl)-pyridin-5-yl]-amide;
4′,6-diisopropyl-biphenyl-2-carboxylic acid-[2-(4-(propen-2-y)l-piperazin-1-yl)-pyridin-5-yl]-amide;
4′,6-Diisopropyl-biphenyl-2-carboxylic acid-[2-(4-(isopropyl)-piperazin-1-yl)-pyridin-5-yl]-amide;
or a physiologically acceptable salt, solvate or derivative thereof.

12. A compound according to any one of claims 1 to 11 for use in therapy.

13. A method for the treatment of a mammal, including man, of conditions ameliorated by an apoB-100 and/or MTP inhibitor comprising administration of an effective amount of a compound according to any one of claims 1 to 11 or a pharmaceutically acceptable derivative thereof.

14. The use of a compound according to any one of claims 1 to 11 or a physiologically acceptable salt or solvate thereof in the manufacture of a medicament for use in the treatment of conditions ameliorated by an apoB-100 and/or MTP inhibitor.

15. A pharmaceutical composition comprising a compound according to any one of claims 1 to 11 or a pharmaceutically acceptable derivative thereof together with one or more pharmaceutically acceptable carriers.

16. A process for the preparation of a compound of formula (I) comprising:
(A) reacting a compound of formula (II) with a compound of formula R′-Z-X-L 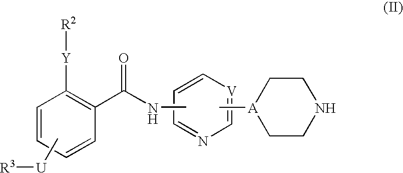
where L represents a suitable halide leaving group, e.g. chloride or bromide, or where X is an oxo group, L may additonally represent a hydroxy group;
(B) reaction of compounds of formula (III) and compounds of formula (VII) 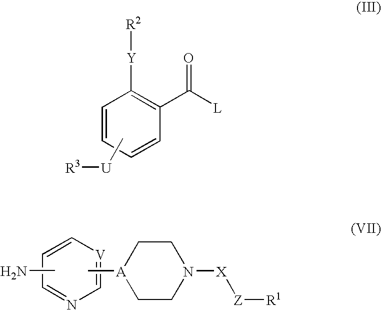
where L is defined above.
(C) reaction of a compound of formula (VIII) with a compound of formula R²—C₁₋₄alkylene-L, where L is defined above; 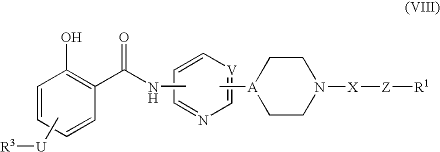
(D) where at least part of X represents an alkylene link to the piperidine or piperazine group, reacting a compound of formula (II) with a compound of formula (IX) 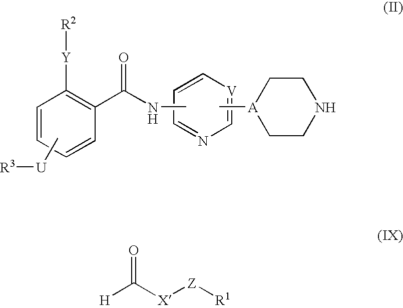
where X′ represents X minus a methylene group; or
(E) reaction of a different compound of formula (I).
